(19) United States
(12) Patent Application Publication
(10) Pub. No.: US 2024/0050715 A1
(43) Pub. Date: Feb. 15, 2024

(54) SEGMENTED MEDICAL BALLOON

(71) Applicant: BOSTON SCIENTIFIC SCIMED, INC., MAPLE GROVE, MN (US)

(72) Inventors: James M. Anderson, Corcoran, MN (US); Gregory Yiu Wah Lee, Eden Prairie, MN (US)

(73) Assignee: BOSTON SCIENTIFIC SCIMED, INC., MAPLE GROVE, MN (US)

(21) Appl. No.: 18/232,037

(22) Filed: Aug. 9, 2023

Related U.S. Application Data
(60) Provisional application No. 63/397,538, filed on Aug. 12, 2022.

(51) Int. Cl. A61M 25/10

(52) U.S. Cl. CPC A61M 25/104; A61M 2025/1072; A61M 2025/1061; A61M 2210/125; A61M 2025/1084

(57) ABSTRACT

Example medical devices are disclosed. An example medical device includes an outer shaft and an inner shaft extending within the outer shaft. The medical device also includes a balloon including a distal waist coupled to a distal end of the inner shaft, a proximal waist coupled to a distal end of the outer shaft, an inner surface and a wall. The medical device also includes a first interior partition panel disposed within the balloon, wherein the first interior partition panel is coupled to both the outer surface of the inner shaft and an inner surface of the balloon and a second interior partition panel disposed within the balloon, wherein the second interior partition panel is coupled to both the outer surface of the inner shaft and an inner surface of the balloon. Further, the first interior partition panel is circumferentially spaced away from the second interior partition panel.

### CROSS-REFERENCE TO RELATED APPLICATIONS

This application claims the benefit of U.S. Provisional Patent Application Ser. No. 63/397,538, filed Aug. 12, 2022, which is incorporated herein by reference.

### TECHNICAL FIELD

The present disclosure relates to methods and apparatus for performing valvuloplasty. More particularly, the present disclosure relates to methods and apparatus for performing valvuloplasty using segmented valvuloplasty balloons.

### BACKGROUND

Heart valve stenosis or calcification is a common manifestation in valvular heart disease, and may often be a leading indicator for balloon valvuloplasty and/or valve replacement therapy. In some instances, balloon valvuloplasty may be beneficial in improving the lifestyle of patients suffering from valve stenosis and may also contribute to a successful valve replacement procedure.

Stenotic or narrowed heart valves may be treated with a number of relatively non-invasive medical procedures including percutaneous transluminal balloon valvuloplasty (PTBV), percutaneous transcatheter heart valve replacement (PTVR) and combinations thereof. Valvuloplasty techniques typically involve advancing a balloon catheter over a guidewire and through an introducer sheath (e.g., an expandable introducer sheath), whereby the valvuloplasty balloon of the balloon catheter is positioned within the heart valve and inflated to dilate the narrowed heart valve.

In other examples, percutaneous transcatheter heart valve replacement (PTVR) may be performed to replace a diseased, native heart valve with an artificial heart valve. One method of performing percutaneous transcatheter heart valve replacement may include the use of a valvuloplasty balloon to dilate the stenotic heart valve prior to implantation of the heart valve.

In yet other examples, a valvuloplasty balloon may be utilized to expand a replacement heart valve placed within a stenotic heart valve. Accordingly, there is an ongoing need for improved valvuloplasty balloons and improved methods of treating valvular heart disease.

### BRIEF SUMMARY

This disclosure provides design, material, manufacturing method, and use alternatives for medical devices. An example medical device includes an outer shaft having a distal end region, an inner shaft extending within the outer shaft, the inner shaft having an outer surface, a proximal end and a distal end. The medical device also includes a balloon including a distal waist coupled to the distal end of the inner shaft, a proximal waist coupled to the distal end of the outer shaft, an inner surface and a wall. The medical device also includes a first interior partition panel disposed within the balloon, wherein the first interior partition panel is coupled to both the outer surface of the inner shaft and an inner surface of the balloon and a second interior partition panel disposed within the balloon, wherein the second interior partition panel is coupled to both the outer surface of the inner shaft and an inner surface of the balloon. Further, the first interior partition panel is circumferentially spaced away from the second interior partition panel.

Alternatively or additionally to any of the embodiments above, wherein the balloon is configured to shift between an inflated configuration and a deflated configuration, and wherein the balloon includes a first inflation chamber positioned between the first interior panel and the second interior panel.

Alternatively or additionally to any of the embodiments above, wherein the balloon is configured to be inflated to a first pressure, and wherein the balloon wall between the first panel and the second panel has a diameter at the first pressure and wherein a portion of the balloon wall attached to the first panel has a diameter at the first pressure, and wherein the diameter of the balloon wall between the first panel and the second panel is greater than the diameter of the portion of the balloon wall attached to the first panel at the first pressure.

Alternatively or additionally to any of the embodiments above, wherein both of the first panel and the second panel extend along a longitudinal axis of the inner shaft.

Alternatively or additionally to any of the embodiments above, wherein the first panel is attached to the inner shaft along a first longitudinal attachment region, and wherein the first panel is substantially perpendicular to a tangent line passing through a point on the first attachment region along the inner shaft.

Alternatively or additionally to any of the embodiments above, wherein the first panel is attached to the inner shaft along a first longitudinal attachment region, and wherein the first panel forms an acute angle relative to a tangent line passing through a point on the first attachment region along the inner shaft.

Alternatively or additionally to any of the embodiments above, wherein the second panel is attached to the inner shaft along a second longitudinal attachment region, and wherein the second panel forms an acute angle relative to a tangent line passing through a point on the second attachment region along the inner shaft.

Alternatively or additionally to any of the embodiments above, wherein the balloon is configured to shift between an inflated configuration and a deflated configuration, and wherein the portions of the balloon wall adjacent a portion of the balloon wall attached to the first panel and the second panel are configured to collapse onto the first panel and the second panel in the deflated configuration.

Alternatively or additionally to any of the embodiments above, wherein the second panel is configured to fold onto the first panel in the deflated configuration.

Alternatively or additionally to any of the embodiments above, wherein the balloon further comprises a third interior partition panel, wherein the third interior partition panel is attached to the outer surface of the inner shaft and an inner surface of the balloon, wherein the third interior partition panel is circumferentially spaced from the second interior partition panel, and wherein the balloon includes a second inflation chamber positioned between the third interior panel and the second interior panel, and wherein the first inflation chamber is configured to be inflated independent of the second inflation chamber.

Another example medical device includes an outer shaft having a distal end region, an inner shaft extending within the outer shaft, the inner shaft having an outer surface, a proximal end and a distal end. The medical device also includes a balloon including a distal waist secured to the distal end of the inner shaft, a proximal waist secured to the distal end of the outer shaft, a wall, and a body portion positioned between the distal waist and the proximal waist. The medical device also includes a first reinforced region positioned along the body portion and a first tension member coupled to the first reinforced region and the inner shaft.

Alternatively or additionally to any of the embodiments above, wherein the first reinforced region includes a first plurality of filaments.

Alternatively or additionally to any of the embodiments above, wherein the first tension member includes a first end region, a second end region and a medial region, and wherein the medial region is wound around the outer surface of the inner shaft.

Alternatively or additionally to any of the embodiments above, wherein the first reinforced region includes a distal end and a proximal end, and wherein the first end region of the first tension member is coupled to the distal end of the reinforced region and wherein the second end region of the first tension member is coupled to the proximal end of the reinforced region.

Alternatively or additionally to any of the embodiments above, wherein the first tension member includes a fiber.

Alternatively or additionally to any of the embodiments above, wherein the balloon is configured to shift between an inflated configuration and a deflated configuration, and wherein the first tension member imparts a radially inward force on the balloon as the balloon shifts between the inflated configuration and the deflated configuration.

Alternatively or additionally to any of the embodiments above, wherein the balloon is configured to shift between an inflated configuration and a deflated configuration, and wherein inflation of the balloon imparts a tensile force on the first tension member.

Alternatively or additionally to any of the embodiments above, further comprising a second reinforced region positioned along the body portion and a second tension member, wherein the second tension member is coupled to the second reinforced region and the inner shaft.

Alternatively or additionally to any of the embodiments above, wherein the second tension member is wound around the inner shaft.

An example method of using a balloon catheter includes advancing a balloon catheter through a body vessel to a target site. The balloon catheter includes an outer shaft having a distal end region, an inner shaft extending within the outer shaft, the inner shaft having an outer surface, a proximal end and a distal end. The balloon catheter also includes a balloon including a distal waist coupled to the distal end of the inner shaft, a proximal waist coupled to the distal end of the outer shaft, an inner surface and a wall. The balloon catheter also includes a first interior partition panel disposed within the balloon, wherein the first interior partition panel is coupled to both the outer surface of the inner shaft and \an inner surface of the balloon. The balloon catheter also includes a second interior partition panel disposed within the balloon, wherein the second interior partition panel is coupled to both the outer surface of the inner shaft and an inner surface of the balloon. Additionally, the first interior partition panel is circumferentially spaced away from the second interior partition panel. The method also includes inflating the balloon, whereby the balloon engages the target site, deflating the balloon and withdrawing the balloon catheter from the body vessel.

The above summary of some embodiments is not intended to describe each disclosed embodiment or every implementation of the present disclosure. The FIGS. and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates a perspective view of an example medical balloon;

FIG. 2A is a cross-sectional view of the medical balloon of FIG. 1 taken along line 2-2 of FIG. 1;

FIG. 2B is a cross-sectional view of another example embodiment of the medical balloon FIG. 1 taken along line 2-2 of FIG. 1;

FIG. 3 is a cross-sectional view of the medical balloon of FIG. 1 taken along line 3-3 of FIG. 1;

FIG. 4 is a partial exploded view of the medical balloon of FIG. 1;

FIG. 5 is a cross-sectional view of the medical balloon of FIG. 1 in an inflated configuration;

FIG. 6 is a cross-sectional view of the medical balloon of FIG. 1 in a deflated configuration;

FIG. 7 is a cross-sectional view of another example medical balloon;

FIG. 8 is a cross-sectional view of another example medical balloon;

FIG. 9 is a perspective view of another example medical balloon;

FIG. 10 is a cross-sectional view of the medical balloon of FIG. 9 taken along line 10-10 of FIG. 9;

FIG. 11 illustrates a perspective view of another example medical balloon;

FIG. 12 is a cross-sectional view of the medical balloon of FIG. 11 taken along line 12-12 of FIG. 11;

FIG. 13 is a cross-sectional view of the medical balloon of FIG. 11 taken along line 13-13 of FIG. 11;

FIG. 14 is a cross-sectional view of the medical balloon of FIG. 11 in a deflated configuration;

FIG. 15 illustrates a perspective view of another example medical balloon;

FIG. 16 is a cross-sectional view of the medical balloon of FIG. 15 taken along line 16-16 of FIG. 15;

FIG. 17 is a cross-sectional view of the medical balloon of FIG. 15 in a deflated configuration.

### DETAILED DESCRIPTION

The following description should be read with reference to the drawings wherein like reference numerals indicate like elements throughout the several views. The detailed description and drawings illustrate example embodiments of the claimed disclosure.

As discussed above, medical balloons may be utilized in a variety of medical treatments. For example, in a valvuloplasty procedure, a valvuloplasty balloon may be used to expand a diseased heart valve. In a percutaneous transcatheter heart valve replacement (PTVR), a valvuloplasty balloon may be used to replace a diseased, native heart valve with an artificial heart valve.

Valvuloplasty balloons may be introduced into a patient by passing through an expandable introducer sheath, through which a guidewire is placed. The valvuloplasty balloon may then be delivered to a target site by advancing the balloon catheter over the guidewire to the target site. In some cases, the pathway to a target site may be tortuous and/or narrow. Upon reaching the site, the valvuloplasty balloon may be expanded by injecting a fluid into the interior of the balloon. Expanding the valvuloplasty balloon may radially expand the stenotic heart valve such that normal blood flow may be restored through the valve.

In some instances, it may be desirable to utilize high pressure valvuloplasty balloons when treating a particular target site (e.g., a stenotic heart valve). To achieve the desired pressure or force against tissue at the target site, a valvuloplasty balloon may be constructed with a thicker balloon wall. However, the thicker balloon wall may increase the profile (e.g., outer diameter) of the balloon when in a deflated configuration. Minimizing the profile of the balloon in a deflated configuration is important as the profile effects the ease and ability of the valvuloplasty balloon to pass through an introducer sheath, through the coronary arteries and across a narrowed heart valve. Further, a reduced profile allows the deflated balloon to pass back through the introducer sheath when the system is removed from the patient. To minimize the outer diameter of the balloon in its deflated condition, it may be desirable to control the folding/refolding mechanics of the valvuloplasty balloon. Examples disclosed herein may include valvuloplasty balloons including partitions, tension members and reinforcing portions designed to control the folding mechanics of the balloon.

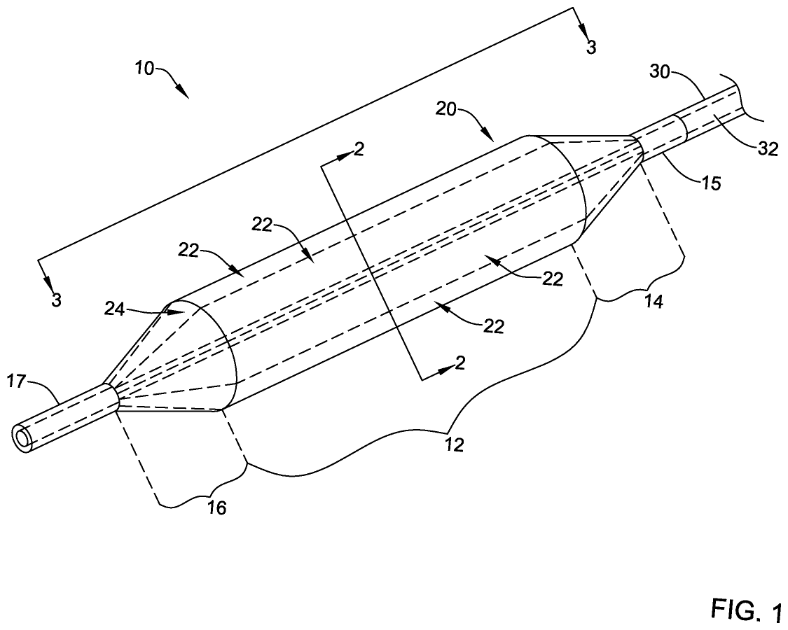

FIG. 1 illustrates an example balloon catheter 10. The balloon catheter 10 may include an expandable medical balloon 20 mounted on or affixed to a distal end of a catheter shaft 30. The medical balloon 20 may be designed to be utilized in a variety of medical procedures, including a valvuloplasty procedure. The catheter shaft 30 may extend from a manifold assembly (not shown) positioned at a proximal end of the catheter shaft 30 and affixed thereto. The balloon 20 may further include a body portion 12, a proximal cone portion 14, a distal cone portion 16, a proximal waist portion 15, and a distal waist portion 17. The body portion 12 may be positioned between the proximal cone portion 14 and the distal cone portion 16, with the proximal waist portion 15 extending proximal of the proximal cone portion 14 and the distal waist portion 17 extending distal of the distal cone portion 16. The balloon 20 may be secured to the catheter shaft 30 at the proximal waist 15. It can be appreciated that the catheter shaft 30 of the balloon catheter 10 may extend through an inner cavity of the balloon 20 and be secured to the distal waist 17 of the balloon 20.

The shaft 30 may include an inner lumen 32. In at least some embodiments, the inner lumen 32 of the shaft 30 may be a guidewire lumen. Accordingly, the catheter 10 may be advanced over a guidewire to the desired location. The guidewire lumen 32 may extend along essentially the entire length of the catheter shaft 30 such that the catheter 10 resembles a traditional “over-the-wire” catheter. Alternatively, the guidewire lumen 32 may extend along only a portion of the shaft 30 so that the catheter 10 resembles a “single-operator-exchange” catheter.

Further, the shaft 30 may also include one or more, or a plurality of inflation lumens that may be used, for example, to transport inflation media to and from the balloon 20. The one or more, or plurality of inflation lumens may be defined within the space between the outer surface of the guidewire lumen 32 and the inner surface of the shaft 30.

The balloon 20 may include a balloon wall constructed of one or more layers, wherein each of the layers may be constructed from different balloon materials. For example, the balloon wall 24 of the balloon 20 may include an inner layer and an outer layer, whereby the inner layer is constructed of a lower durometer (e.g., softer) material as compared to the outer layer. This two-layer construction may be referred to as a bi-layer balloon base layer. Further, the inner and outer layer of the bi-layer balloon wall 24 may be co-extruded during the manufacturing process of the balloon. Typical balloon materials may include polymer materials, some examples of which are listed herein.

Additionally, FIG. 1 illustrates that the balloon 20 may include one or more interior partitioning panels (e.g., radially extending interior fins, ribs, columns, walls, partitions, etc.) partitioning the interior of the balloon 20 into a plurality of discrete inflation chambers. For example, the balloon 20 may include one, two, three, four, five, six, seven, eight or more interior partitioning panels 22 (e.g., radially extending interior fins, ribs, columns, walls, partitions, etc.) circumferentially arranged around the longitudinal axis of the balloon 20. The balloon 20 illustrated in FIG. 1 includes eight panels 22 (it can be appreciated that multiple panels 22 are hidden from view in FIG. 1).

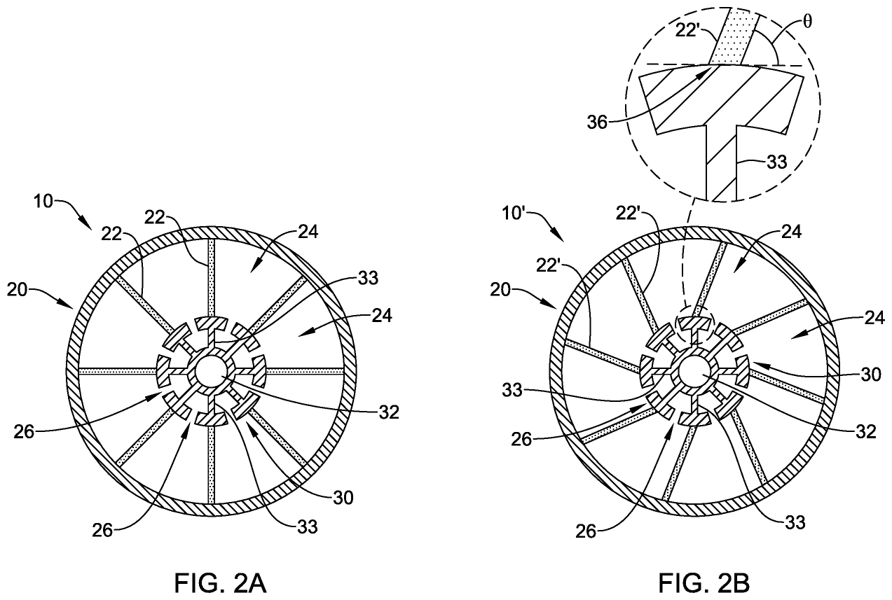

In some examples, each of the panels 22 may extend between and be secured to both the shaft 30 and the balloon 20. For example, each of the panels 22 may include a first end or first longitudinal extent which is attached to the outer surface of the shaft 30. Additionally, each of the panels 22 may include a second end or second longitudinal extent which is attached to the inner surface of the balloon 20 along the distal cone region 16, the body portion 12 and the proximal cone region 14. As will be described in greater detail with respect to FIGS. 2A and 2B, it can be appreciated that the shaft 30, the balloon 20 and any two adjacent interior partitioning panels 22 may define an inner chamber 24 positioned between the adjacent panels 22. In other words, each panel 22 may be sealed along the outer surface of the shaft 30 and along the inner surface of the balloon 20 such that each chamber 24 defines a separate and distinct inflation region of the balloon 20. Each distinct inflation chamber 24 may be fluidly isolated from one or more of the other inflation chambers 24 within the balloon 20. For example, in some instances each distinct inflation chamber 24 may be fluidly isolated from each of the other inflation chambers 24 within the balloon 20.

In some examples, a manifold attached to the outer shaft 20 may be configured to allow selective inflation of one or more of the chambers 24. In other words, the balloon catheter 10 may include a manifold which permits inflation of each chamber 24 independently of one or more of the other chambers 24. In some instances, the balloon catheter 10 may include a manifold which permits inflation of each chamber 24 independently of each of the other chambers 24. Accordingly, in the event a single chamber 24 fails to inflate, one or more other chambers 24 may still be inflated despite the failure of the single chamber 24 to inflate.

FIG. 2A is a cross-sectional view of the medical balloon of FIG. 1 taken along line 2-2 of FIG. 1. FIG. 2A illustrates the interior partitioning panels 22 circumferentially disposed around the outer surface of the shaft 30. Each of the panels 22 may include a first end or first extent which is attached to the outer surface of the shaft 30 and may also include a second end or second extent which is attached to the inner surface of the distal cone region 16, the body portion 12, and the proximal cone region 14. It can be appreciated that each of the panels 24 may be a monolithic structure connected between the shaft 30 and the balloon 20 that extends radially outward from the outer surface of the shaft 30 to the inner surface of the balloon 20, thereby forming individual panels (e.g., walls, ribs, etc.) which are circumferentially spaced from one another, as described herein.

Additionally, FIG. 2A illustrates that the catheter shaft 30 may also include a plurality of inflation lumens defined between one or more rib members 33 extending longitudinally from a distal end region of the catheter shaft 30 to a manifold which may be attached to a proximal end of the catheter shaft 30. It can be appreciated that each of the rib members 33 may establish multiple, distinct inflation lumens. For example, FIG. 2A illustrates the rib members 33 may divide the catheter shaft 30 into eight separate and distinct inflation lumens.

As described above, FIG. 2A illustrates that any two adjacent panels 22 may define a distinct inflation chamber positioned between two adjacent panels 22. FIG. 2A further illustrates that the balloon 20 may include a plurality of apertures 26 in fluid communication with the one or more distinct inflation lumens of the catheter shaft 30. For instance, the catheter may include at least one aperture 26 located in each of the distinct inflation lumens, whereby at least one aperture 26 may be in fluid communication with each of the inflation chambers 24, thus permitting inflation fluid to pass from an inflation lumen of the catheter shaft 30 into the respective inflation chamber 24. The apertures 26 may be circumferentially aligned with each of the inflation chambers 24, such that the apertures 26 may permit an inflation media (e.g., inflation fluid) to pass from an inflation lumen into the inflation chambers 24. As described above, the balloon catheter 10 may include a manifold which permits the inflation of each chamber 24 independently of the other chambers 24. For example, the balloon catheter 10 may include one or more independent inflation lumens in fluid communication with a respective inflation chamber 24 which permit, via the manifold, the independent inflation of one or more of the chambers 24. As discussed above, each inflation chamber 24 may be in fluid communication with a separate, dedicated inflation lumen extending through the catheter shaft 30. Accordingly, in the event a single chamber 24 fails to inflate, one or more other chambers 24 may still be inflated despite the failure of a single chamber 24 to inflate.

FIG. 2B is a cross-sectional view of another example medical balloon catheter 10′. The medical balloon catheter 10′ may be similar in form and function to the medical balloon catheter 10 described above. For example, the medical balloon catheter 10′ may include the catheter shaft 30 positioned within the balloon body portion 12 as described above. Further, the cross-sectional view shown in FIG. 2B illustrates the balloon 20 may include one or more interior partitioning panels 22′ circumferentially spaced around the outer surface of the catheter shaft 30, the partitioning panels 22′ dividing the balloon 20 into a plurality of discrete inflation chambers 24. Each distinct inflation chamber 24 may be fluidly isolated from one or more of the other inflation chambers 24 within the balloon 20. For example, in some instances each distinct inflation chamber 24 may be fluidly isolated from each of the other inflation chambers 24 within the balloon 20.

The interior partitioning panels 22′ may be similar in form and function to the interior panels 22 described herein. Each of the panels 22′ may extend between and be secured to both the shaft 30 and the wall of the balloon 20. For example, each of the panels 22′ may include a first end or first extent which is attached to the outer surface of the catheter shaft 30 and may also include a second end or second extent which is attached to the inner surface of the proximal cone region 14, the body portion 12, and the distal cone region 16. It can be appreciated that each of the panels 22′ may be a monolithic structure that extends radially outward from the outer surface of the catheter shaft 30 to the inner surface of the proximal cone region 14, the body portion 12, and the distal cone region 16, thereby forming individual panels 22′ (e.g., fins, ribs, columns, walls, partitions, etc.) which are circumferentially spaced from one another, as described herein.

Further, FIG. 2B illustrates that the balloon 20 may include a plurality of apertures 26 in fluid communication with one or more inflation lumens of the balloon catheter 10. For instance, the catheter 10 may include at least one aperture 26 in fluid communication with each of the inflation chambers 24, thus permitting inflation fluid to pass from an inflation lumen of the catheter shaft 30 into the respective inflation chamber 24. The apertures 26 may be circumferentially aligned with each of the inflation chambers 24, such that the apertures 26 may permit an inflation media (e.g., inflation fluid) to pass from an inflation lumen into the inflation chambers 24. As described above, the balloon catheter 10′ may include a manifold which permits the inflation of each chamber 24 independently of one or more of the other chambers 24. In some instances, the balloon catheter 10 may include a manifold which permits inflation of each chamber 24 independently of each of the other chambers 24. For example, the balloon catheter 10′ may include one or more independent inflation lumens which permit, via the manifold, the independent inflation of one or more of the chambers 24. Accordingly, in the event a single chamber 24 fails to inflate, one or more other chambers 24 may still be inflated despite the failure of a single chamber 24 to inflate.

FIG. 2B further illustrates that one or more of the panels 22′ may extend from the outer surface of the catheter shaft 30 at an angle such that the plurality of panels 22′ may form an iris-type configuration around the catheter shaft 30. For example, the detailed view of FIG. 2B illustrates that each of the panels 22′ may form an acute angle θ with respect to a tangent line 34 running through the attachment point 36 of a panel 22′ to the outer surface of the catheter shaft 30.

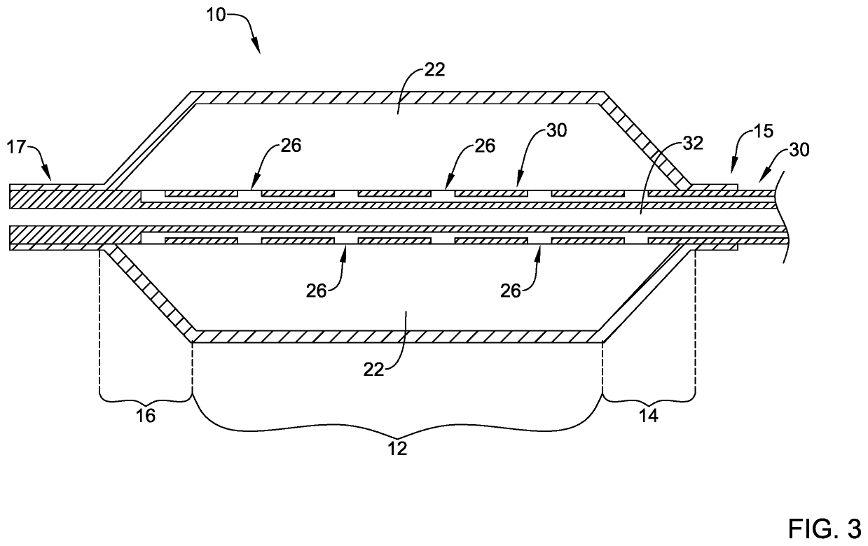

FIG. 3 is a cross-sectional view of the medical balloon catheter 10 taken along line 3-3 of FIG. 1. FIG. 3 illustrates two interior portioning panels 22, which are circumferentially offset 180 degrees from one another. As described herein, each of the panels 22 may include a first longitudinal extent which is attached to the outer surface of the catheter shaft 30 (e.g., a first edge of each of the panels is attached to and extends longitudinally along an outer surface of the catheter shaft 30). Additionally, each of the panels 22 may include a second longitudinal extent which is attached to the inner surface of the balloon 20 and extends along the proximal cone region 14, the body portion 12 and the distal cone region 16 (e.g., a second edge of each of the panels is attached to and extends longitudinally along an inner surface of the proximal cone region 14, the body portion 12 and the distal cone region 16).

Additionally, as described herein, FIG. 3 illustrates that the catheter shaft 30 may include a plurality of apertures 26 in fluid communication with one or more inflation lumens of the balloon catheter 10. As illustrated in FIGS. 2A-2B, one or more of the apertures 26 may be circumferentially aligned with each of the inflation chambers 24 defined by any two adjacent panels 22. Further, FIG. 3 illustrates that the apertures 26 may also extend along the longitudinal axis of the balloon 20, such that a plurality of longitudinally aligned apertures 26 are in fluid communication with each of the inflation chambers 24. Accordingly, each individual inflation chamber 24 (defined between any two panels 22) may be inflated via a plurality of apertures 26 aligned with one another along the longitudinal axis of the balloon 20.

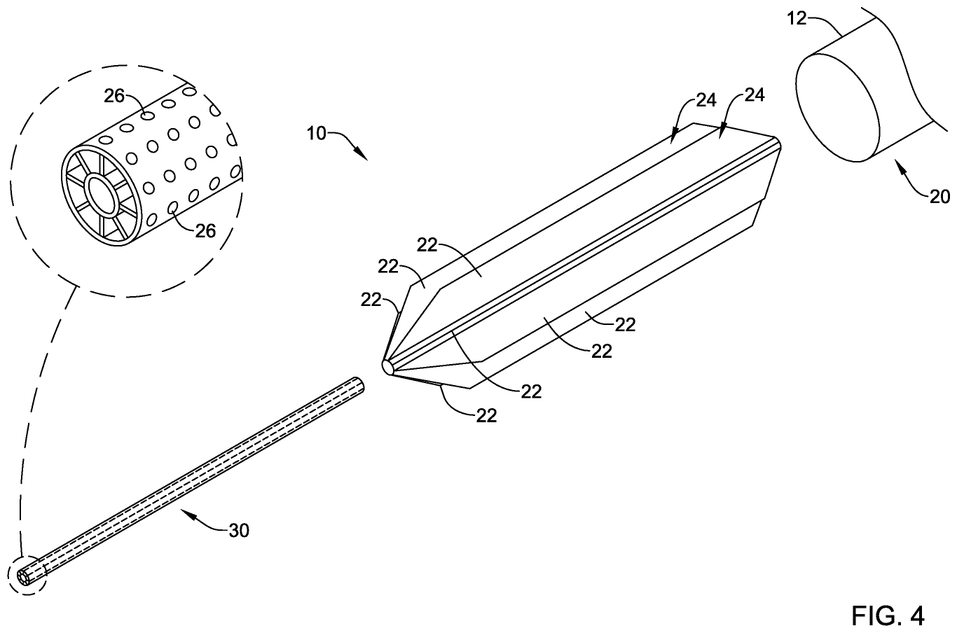

FIG. 4 illustrates an exploded view of various components of the balloon catheter 10. For example, FIG. 4 illustrates the catheter shaft 30, the plurality of circumferentially spaced interior partitioning panels 22 and a portion of the body 12 of the balloon 20. FIG. 4 illustrates that the plurality of interior partitioning panels 22 may be evenly spaced from one another around the circumference of the catheter shaft 30. Accordingly, FIG. 4 illustrates that the balloon catheter 10 may include eight panels 22 (a single panel 22 is hidden from view in FIG. 4) which define eight inflation chambers 24 (a single inflation chamber 24 defined between two adjacent panels 22). In some examples, the balloon catheter 10 may include fewer or greater than eight partitioning panels 22. For example, the balloon catheter 10 may include 2, 3, 4, 5, 6, 7, 8, 9, 10, or more partition panels and associated inflation chambers. FIG. 4 further illustrates that each of the panels 22 may include an outermost edge (in some instances referred to herein as a second extent) which is configured to match the shape (e.g., contour, geometry, etc.) of the inner surface of the balloon 20. For example, it can be appreciated from FIG. 4 that each of the panels 22 may be shaped to match and form a seal with inner surface of the proximal cone 14, the body portion 12 and distal cone 16 of the balloon 20 illustrated in FIG. 1. In some instances, the panels 22 may be formed integrally with the body portion 12, such that the panels 22 are a monolithic structure of the body portion 12 of the balloon 20. In other instances, the panels 22 may be formed separately and then fixed to the body portion 12 of the balloon 20.

In some examples, the balloon catheter 10 described herein may be constructed by extruding the interior partitioning panels 22 in conjunction with the extrusion of the balloon 20. It can be appreciated that to complete the construction of the balloon catheter 10, the catheter shaft 30 may then be inserted into the center of the panels 22 whereby each of the panels 22 are attached (via any suitable attachment technique including, but not limited to, adhesive bonding, laser welding, etc.) to the outer surface of the catheter shaft 30. In other manufacturing methods, each of the panels 22 may be individually attached to the outer surface of the catheter shaft 30 to form a catheter shaft/panel subassembly, whereby the catheter shaft/panel subassembly is then inserted into and attached to inner surface of the balloon 20. In yet other manufacturing methods, each of the panels 22 may be attached (via any suitable attachment technique including, but not limited to, adhesive bonding, laser welding, etc.) to the inner surface of the balloon and then attached to the catheter shaft 30. Further, in some examples, a channel may be formed along an inner surface of the balloon in which a panel 22 may be positioned. After the panel 22 has been positioned in the channel, the balloon and the panel 22 may be heat bonded together.

The detailed view of FIG. 4 illustrates that the catheter shaft 30 may include one or more of the apertures 26 which are circumferentially aligned with each of the inflation chambers 24 defined by any two adjacent panels 22 of the balloon 20. Further, FIG. 4 illustrates that the apertures 26 may also extend along the longitudinal axis of the catheter shaft 30, such that the plurality of longitudinally aligned apertures 26 are in fluid communication with each of the inflation chambers 24. Accordingly, each individual inflation chamber 24 (defined between any two panels 22) may be inflated via a plurality of apertures 26 aligned with one another along the longitudinal axis of the balloon 20.

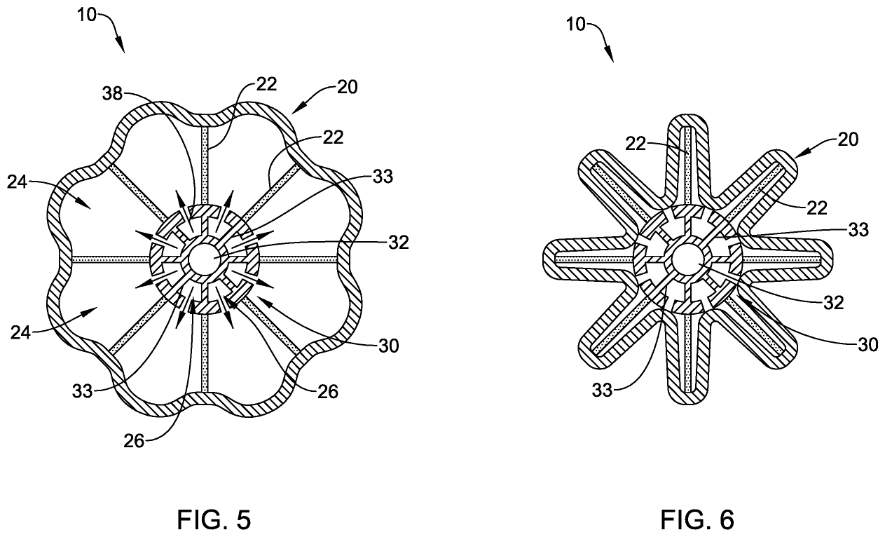

FIG. 5 illustrates a cross-sectional view of the balloon 20 of the balloon catheter 10 in which an inflation media (e.g., inflation fluid) is being transported through multiple inflation lumens of the catheter 10, passing through the apertures 26 and inflating the respective chambers 24 defined between adjacent panels 22. The arrows 38 of FIG. 5 illustrate the inflation fluid passing from the inflation lumens into the individual chambers 24. As described herein, it can be appreciated that, in some examples, the balloon catheter 10 may include a manifold which is configured to permit selective inflation of one or more of the inflation chambers 24. Accordingly, in instances in which one or more of the inflation chambers 24 fails to inflate, the remaining chambers 24 may continue to be inflated.

FIG. 5 further illustrates that during the inflation of the balloon 20, each individual panel 22 may be constructed such that the panel prevents a portion of the balloon 20 wall from extending radially outward. For example, as depicted in FIG. 5, as the balloon 20 is inflated, portions of the balloon wall which extend between any two panels 22 may bulge radially outward beyond the radial extent of the individual panels 22. It can be appreciated that, when inflated, the balloon 20 material positioned between any two points at which the balloon material is attached to a panel 22 may define the greatest radial extent of the balloon 20. In other words, it can be appreciated that, during inflation, each of the panels 22 may be strong enough to withstand the radial inflation forces of the balloon 20, and therefore, each of the panels 22 may prevent a portion of the balloon 20 located directly radially outward of the corresponding panel 22 from expanding radially outward, and thereby permitting the portions of the balloon 20 positioned between any two adjacent panels 22 to extend radially outward (e.g., bulge outward) relative to the portions of the balloon 20 attached to the panels 22. Such a configuration may provide a balloon 20 having a plurality of longitudinal flutes or valleys arranged around the outer circumference of the balloon 20 between adjacent longitudinal ridges or peaks.

FIG. 6 illustrates the balloon 10 described in FIG. 5 after vacuum (i.e., negative pressure) has been pulled to deflate the balloon 20. It can be appreciated that as vacuum is pulled on the inflation media, the wall of the balloon 20 may suck down on to each of the panels 22 and/or draw inward against the panels 22. FIG. 6 shows that the panels 22 may be constructed with sufficient column strength to maintain their relative shape despite the radial collapse of the balloon 20 on to the panels 22. It can be further appreciated that designing the panels 22 to have enough column strength to maintain their relative shape during the deflation of the balloon 20 may result in a predictable, deflated, cross-sectional shape of the balloon 20.

As illustrated in FIG. 6, the cross-sectional shape of the deflated balloon 20 forms a relatively uniform distribution of the balloon material, whereby a substantially uniform amount of balloon material is positioned along each of the panels 22 in the deflated configuration. Further, it can be appreciated that the uniform distribution of balloon material along the panels 22 may permit the balloon 20 to refold in a more efficient, symmetrical and uniform configuration. In other words, the configuration of the panels 22 and subsequent deflated configuration of the balloon 20 (as shown in FIG. 6) may facilitate a refolded balloon 20 which includes a reduced outer profile (e.g., reduced overall outer diameter) in a deflated configuration. It can be appreciated a reduced outer profile may be beneficial when during the withdraw of the deflated balloon 20 into an introducer sheath.

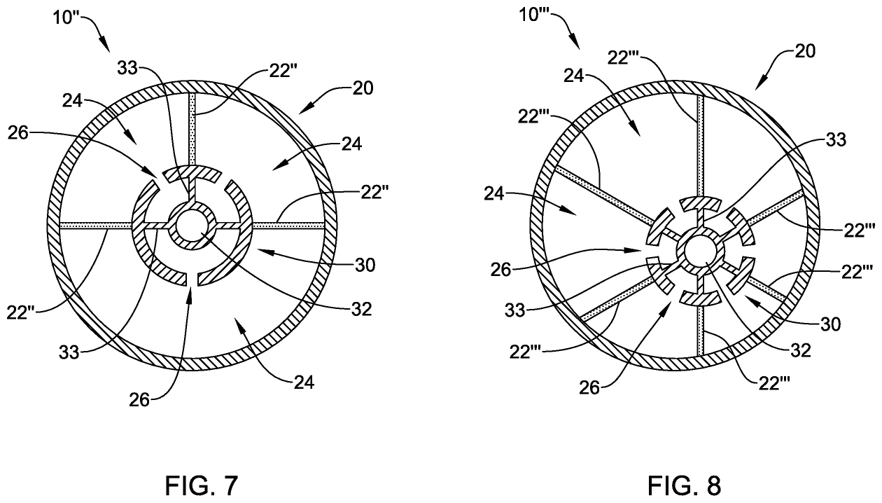

FIG. 7 is a cross-sectional view of another example medical balloon catheter 10″. The medical balloon catheter 10″ may be similar in form and function to the medical balloon catheter 10 described above. For example, the medical balloon catheter 10″ may include the catheter shaft 30 positioned within the balloon body portion 12, as described herein. Further, FIG. 7 illustrates the balloon 20 may include one or more interior partitioning panels 22″ spaced around the catheter shaft 30 dividing the balloon 20 into a plurality of discrete inflation chambers 24. Each distinct inflation chamber 24 may be fluidly isolated from one or more of the other inflation chambers 24 within the balloon 20. For example, in some instances each distinct inflation chamber 24 may be fluidly isolated from each of the other inflation chambers 24 within the balloon 20.

The interior partitioning panels 22″ may be similar in form and function to the interior panels 22 described herein. Each of the panels 22″ may extend between and be secured to both the catheter shaft 30 and the wall of the balloon 20. For example, each of the panels 22″ may include a first end or first extent which is attached to the outer surface of the catheter shaft 30 and may also include a second end or second extent which is attached to the inner surface of the balloon 20 (along the proximal cone region 14, the body portion 12, and the distal cone region 16). It can be appreciated that each of the panels 22″ may be a monolithic structure that extends radially outward from the outer surface of the catheter shaft 30 to the inner surface of the balloon 20, thereby forming panels 22″ (e.g., fins, ribs, columns, walls, partitions, etc.) which are circumferentially spaced from one another, as described herein.

Further, FIG. 7 illustrates that the catheter shaft 30 may include a plurality of apertures 26 in fluid communication with one or more inflation lumens of the catheter 10″. For instance, the catheter 10″ may include at least one aperture 26 in fluid communication with each of the inflation chambers 24, thus permitting inflation fluid to pass from an inflation lumen of the catheter 10″ into the respective inflation chamber 24. As illustrated in FIG. 7, the apertures 26 may be circumferentially aligned with each of the inflation chambers 24 defined by any two adjacent panels 22″. The apertures 26 may be circumferentially aligned with each of the inflation chambers 24, such that the apertures 26 may permit an inflation media (e.g., inflation fluid) to pass from an inflation lumen of the balloon catheter 10″ into the inflation chambers 24. As described above, the balloon catheter 10″ may include a manifold which permits the inflation of each chamber 24 independently of one or more of the other chambers 24. In some instances, the balloon catheter 10″ may include a manifold which permits inflation of each chamber 24 independently of each of the other chambers 24.

FIG. 7 further illustrates that the balloon catheter 10″ may include interior partitioning panels 22″ arranged around the catheter shaft 30 to form three distinct inflation chambers 24. As shown in FIG. 7, the panels 22″ may be arranged around the catheter shaft 30 to form inflation chambers 24 having different sizes (e.g., one or more of the inflation chambers 24 of the balloon 10″ may be sized differently than the other chambers 24). For example, FIG. 7 illustrates that the balloon catheter 10″ may be designed to include three panels 22″ distributed around the catheter shaft 30 in a configuration that defines two inflation chambers 24 which are substantially uniform in size and a third inflation chamber 24 that is larger than each of the other uniformly-sized inflation chambers 24.

FIG. 8 is a cross-sectional view of another example medical balloon catheter 10′″. The medical balloon catheter 10′″ may be similar in form and function to the medical balloon catheter 10 described above. For example, the medical balloon catheter 10′″ may include the catheter shaft 30 positioned within balloon body portion 12 as described above. Further, the cross-sectional view shown in FIG. 8 illustrates the balloon 20 may include one or more interior partitioning panels 22′″ spaced around the outer surface of the catheter shaft 30 dividing the balloon 20 into a plurality of discrete inflation chambers 24. Each distinct inflation chamber 24 may be fluidly isolated from one or more of the other inflation chambers 24 within the balloon 20. For example, in some instances each distinct inflation chamber 24 may be fluidly isolated from each of the other inflation chambers 24 within the balloon 20.

The interior partitioning panels 22′″ may be similar in form and function to the interior panels 22 described herein. Each of the panels 22′″ may extend between and be secured to both the catheter shaft 30 and the wall of the balloon 20. For example, each of the panels 22′″ may include a first end or first extent which is attached to the outer surface of the catheter shaft 30 and may also include a second end or second extent which is attached to the inner surface of the balloon 20 (along the proximal cone region 14, the body portion 12, and the distal cone region 16). It can be appreciated that each of the panels 22′″ may be a monolithic structure that extends radially outward from the outer surface of the catheter shaft 30 to the inner surface of the balloon 20, thereby forming individual panels 22′″ (e.g., fins, ribs, columns, walls, partitions, etc.) which are circumferentially spaced from one another, as described herein.

FIG. 8 illustrates that the catheter shaft 30 may include a plurality of apertures 26 in fluid communication with one or more inflation lumens of the balloon catheter 10′″. For instance, the catheter 10′″ may include at least one aperture 26 in fluid communication with each of the inflation chambers 24, thus permitting inflation fluid to pass from an inflation lumen 32 of the catheter 10′″ into the respective inflation chamber 24. As illustrated in FIG. 8, the apertures 26 may be circumferentially aligned with each of the inflation chambers 24 defined by any two adjacent panels 22′″. The apertures 26 may be circumferentially aligned with each of the inflation chambers 24, such that the apertures 26 may permit an inflation media (e.g., inflation fluid) to pass from an inflation lumen of the balloon catheter 10′″ into the inflation chambers 24. As described above, the balloon catheter 10′″ may include a manifold which permits the inflation of each chamber 24 independently of one or more of the other chambers 24. In some instances, the balloon catheter 10′″ may include a manifold which permits inflation of each chamber 24 independently of each of the other chambers 24.

FIG. 8 further illustrates that the balloon catheter 10′″ may include interior partitioning panels 22′″ dividing the interior of the balloon 20 into a plurality of inflation chambers 24, whereby one or more of the interior partitioning panels 22′″ extends away from the catheter shaft 30 a greater distance compared to one or more other of the interior partitioning panels 22′″. In other words, one or more of the partitioning panels 22′″ may have a greater radial height than others of the partitioning panels 22′″. In the example of FIG. 8, one or more of the partitioning panels 22′″ extends away from the outer surface of the catheter shaft 30 a greater distance compared to other panels 22′″. It can be appreciated from FIG. 8 that this configuration results in the central longitudinal axis of the catheter shaft 30 being offset from the central longitudinal axis of the balloon 20. Further, FIG. 8 illustrates a configuration in which the inflation chambers 24 (defined by adjacent panels 22′″) may all be different sizes. For example, FIG. 8 illustrates that the balloon 10′″ may be designed to include six panels 22′″, whereby each of the panels 22′″ has a different radial extent and are distributed around the catheter shaft 30 in a configuration that defines six inflation chambers 24 which are different sizes relative to one another.

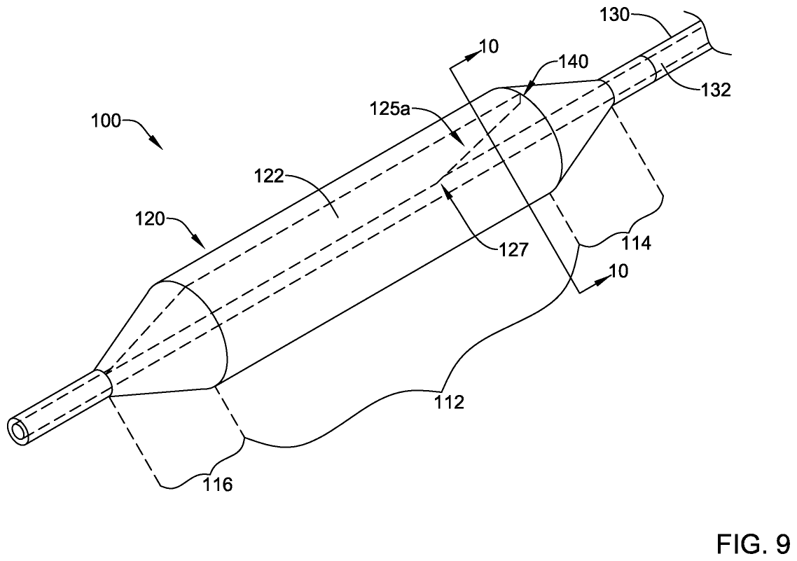

FIG. 9 illustrates another example balloon catheter 100. The balloon catheter 100 may include an expandable medical balloon 120 mounted on or affixed to a catheter shaft, such as a distal end of an outer catheter shaft 130. The medical balloon 120 may be designed to be utilized in a variety of medical procedures, including a valvuloplasty procedure. The catheter shaft 130 may extend from a manifold assembly (not shown) positioned at a proximal end of the catheter shaft 130 and affixed thereto. The balloon 120 may further include a body portion 112, a proximal cone portion 114, a distal cone portion 116, a proximal waist portion 115, and a distal waist portion 117. The body portion 112 may be positioned between the proximal cone portion 114 and the distal cone portion 116, with the proximal waist portion 115 extending proximal of the proximal cone portion 114 and the distal waist portion 117 extending distal of the distal cone portion 116. The balloon 120 may be secured to the outer catheter shaft 130 at the proximal waist 115. It can be appreciated that the catheter shaft 130 of the balloon catheter 110 may extend through an interior of the balloon 120 and be secured to the distal waist 117 of the balloon 120.

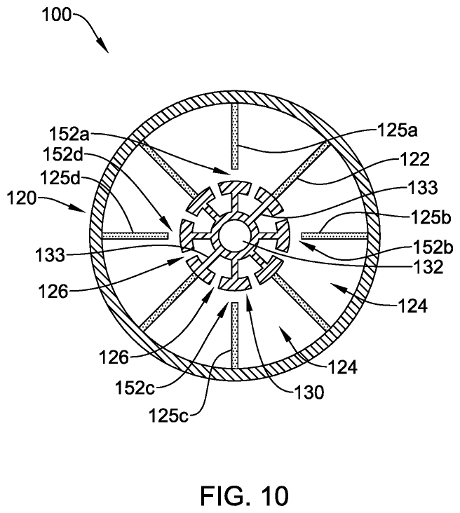

FIG. 9 further illustrates that the medical balloon 120 may include one or more interior partitioning panels 122 (e.g., radially extending interior fins, ribs, columns, walls, partitions, etc.) circumferentially arranged around the longitudinal axis of the balloon 120 and partitioning the interior of the balloon 120 into a plurality of discrete inflation chambers. The panels 122 may be similar in form and function to the panels 22 described herein. For simplicity, FIG. 9 illustrates the catheter 100 including two panels 122. However, it can be appreciated that the balloon 120 may include one, two, three, four, five, six, seven, eight or more panels 122. For example, FIG. 10, which is a cross-sectional view of the balloon catheter 100 taken along line 10-10 of FIG. 9, illustrates that the balloon 120 may include eight interior partitioning panels 122, 125a, 125b, 125c, 125d (e.g., radially extending interior fins, ribs, columns, walls, partitions, etc.) circumferentially arranged around the longitudinal axis of the balloon 120.

Similarly, to that described herein with respect to the panels 22 of the balloon 20, each of the panels 122 may include a first end or first longitudinal extent which is attached to the outer surface of the catheter shaft 130. Additionally, each of the panels 122 may include a second end or second longitudinal extent which is attached to the inner surface of the balloon 120 and extends along the distal cone region 116, the body portion 112 and the proximal cone region 114. Accordingly, as illustrated in FIG. 10, it can be appreciated that any two adjacent interior partitioning panels 122 may define an inner chamber 124 positioned between the adjacent panels 122. In other words, each panel 122 may be sealed along the outer surface of the catheter shaft 130 and along the inner surface of the distal cone region 116, the body portion 112 and the proximal cone region 114 such that each chamber 124 defines a separate and distinct inflation region of the balloon 120. Each distinct inflation chamber 124 may be fluidly isolated from one or more of the other inflation chambers 124 within the balloon 120. For example, in some instances each distinct inflation chamber 124 may be fluidly isolated from each of the other inflation chambers 124 within the balloon 120.

Further, FIGS. 9-10 illustrate that the example balloon 120 may include one or more panels 122a, 122b, 122c, 122d having a proximal end region 152a, 152b, 152c, 152d which is unattached to the outer surface of the catheter shaft 130. For example, FIG. 9 illustrates a panel 122 which includes a proximal end region 125 that is unattached to the outer surface of the catheter shaft 130 (FIG. 9 illustrates that the panel 122 is unattached from the outer surface of the catheter shaft 130 between the attachment point 127 and the proximal end 140 of the proximal end region 125 of the panel 122).

As described above, FIG. 10 illustrates the interior partitioning panels 122 circumferentially disposed around the outer surface of the catheter shaft 130. FIG. 10 illustrates that any two adjacent panels 122 may define a distinct inflation chamber 124 positioned between two adjacent panels 122. FIG. 10 further includes a plurality of apertures 126 in fluid communication with one or more inflation lumens of the balloon catheter 100. For instance, the catheter 100 may include at least one aperture 126 in fluid communication with each of the inflation chambers 124, thus permitting inflation fluid to pass from an inflation lumen of the catheter 100 into the respective inflation chamber 124. The apertures 126 may be circumferentially aligned with each of the inflation chambers 124, such that the apertures 126 may permit an inflation media (e.g., inflation fluid) to pass from an inflation lumen into the inflation chambers 124. As described herein, the balloon catheter 100 may include a manifold which permits the inflation of each chamber 124 independently of the other chambers 124. For example, similarly to that described above with respect to the catheter 10, the balloon catheter 100 may include one or more independent inflation lumens in fluid communication with a respective inflation chamber 124 which permit, via the manifold, the independent inflation of one or more of the chambers 124. Thus, each inflation chamber 124 may be in fluid communication with a separate, dedicated inflation lumen extending through the catheter shaft. Accordingly, in the event a single chamber 124 fails to inflate, one or more other chambers 124 may still be inflated despite the failure of a single chamber 124 to inflate.

FIG. 10 further illustrates the proximal end region 125 of the panel 122 unattached to the outer surface of the catheter shaft 130. FIG. 10 illustrates the gap 152 radially between the panel 122 and the outer surface of the catheter shaft 130. It can be appreciated that when the balloon 120 is inflated, the portion of the balloon wall (depicted in FIG. 10 as the balloon wall “X”) adjacent to proximal end region 125 of the panel 122 may expand radially outward to a greater extent than other portions of the balloon wall (e.g., the portion of the balloon 120 which is adjacent to the unattached, proximal end region 125 of the panel 122 is not restrained from expanding radially outward compared to portions of the balloon 120 which are coupled to attached panels 122).

With regard to the apertures of any of the preceding embodiments, the plurality of apertures 26, 126 in communication with any one of the chambers 24, 124 may be equidistantly arranged longitudinally along the catheter shaft 30, 130, or the apertures 26, 126 may be otherwise longitudinally arranged. For example, there may be more apertures 26, 126, larger sized (e.g., larger diameter) apertures 26, 126, and/or more closely arranged apertures 26,126 closer to the distal end of the catheter shaft, and thus closer to a distal end region of the chambers 24, 124 in some instances. Such a configuration may assist in inflating a distal end region of the balloon 20, 120 relative to a proximal end region of the balloon 20, 120. In other instances, there may be more apertures 26, 126, larger sized (e.g., larger diameter) apertures 26, 126, and/or more closely arranged apertures 26,126 closer to the proximal end of the catheter shaft, and thus closer to a proximal end region of the chambers 24, 124 in some instances. Such a configuration may assist in inflating a proximal end region of the balloon 20, 120 relative to a distal end region of the balloon 20, 120. In yet other instances, there may be more apertures 26, 126, larger sized (e.g., larger diameter) apertures 26, 126, and/or more closely arranged apertures 26,126 along the medial region of the chambers 24, 124 relative to the proximal and distal end regions of the chambers 24, 124 in some instances. Such a configuration may assist in inflating a medial region of the balloon 20, 120 relative to the proximal and distal end regions of the balloon 20, 120.

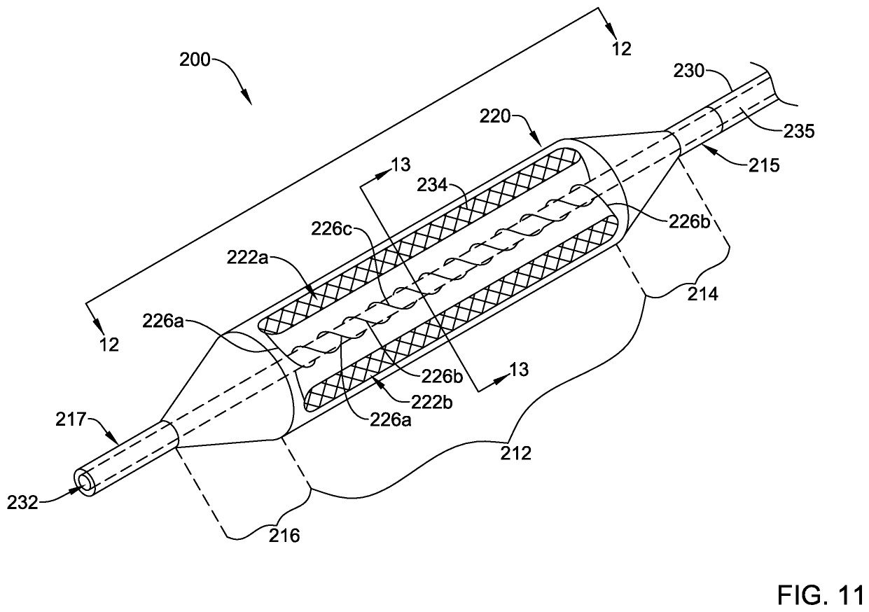

FIG. 11 illustrates an example balloon catheter 200. The balloon catheter 200 may include an expandable medical balloon 220 mounted on or affixed to a distal end of a catheter shaft, such as an outer catheter shaft 230. The medical balloon 220 may be designed to be utilized in a variety of medical procedures, including a valvuloplasty procedure. The catheter shaft 230 may extend from a manifold assembly (not shown) positioned at a proximal end of the catheter shaft 230 and affixed thereto. The balloon 220 may further include a body portion 212, a proximal cone portion 214, a distal cone portion 216, a proximal waist portion 215, and a distal waist portion 217. The body portion 212 may be positioned between the proximal cone portion 214 and the distal cone portion 216, with the proximal waist portion 215 extending proximal of the proximal cone portion 214 and the distal waist portion 217 extending distal of the distal cone portion 216. The balloon 220 may be secured to the outer catheter shaft 230 at the proximal waist 215. It can be appreciated that the catheter shaft of the balloon catheter 200 may also include an inner shaft 232 that may extend within the outer catheter shaft 230, through an inner cavity of the balloon 220 and be secured to the distal waist 217 of the balloon 220.

The inner shaft 232 may include an inner lumen. In at least some embodiments, the inner lumen of the inner shaft 232 may be a guidewire lumen. Accordingly, the catheter 200 may be advanced over a guidewire to the desired location. The guidewire lumen may extend along essentially the entire length of the catheter shaft 230 such that the catheter 200 resembles a traditional “over-the-wire” catheter. Alternatively, the guidewire lumen may extend along only a portion of the shaft 230 so that the catheter 200 resembles a “single-operator-exchange” catheter.

Further, the outer shaft 230 may also include an inflation lumen that may be used, for example, to transport inflation media to and from the balloon 220. When the outer shaft 230 is disposed over the inner shaft 232, the inflation lumen may be defined within the space between the outer surface of the inner shaft 232 and the inner surface of the outer shaft 230.

The balloon 220 may include a balloon wall constructed of one or more layers, wherein each of the layers may be constructed from different balloon materials. For example, the balloon wall 224 of the balloon 220 may include an inner layer and an outer layer, whereby the inner layer is constructed of a lower durometer (e.g., softer) material as compared to the outer layer. This two-layer construction may be referred to as a bi-layer balloon base layer. Further, the inner and outer layer of the bi-layer balloon wall 224 may co-extruded during the manufacturing process of the balloon. Typical balloon materials may include polymer materials, some examples of which are listed herein.

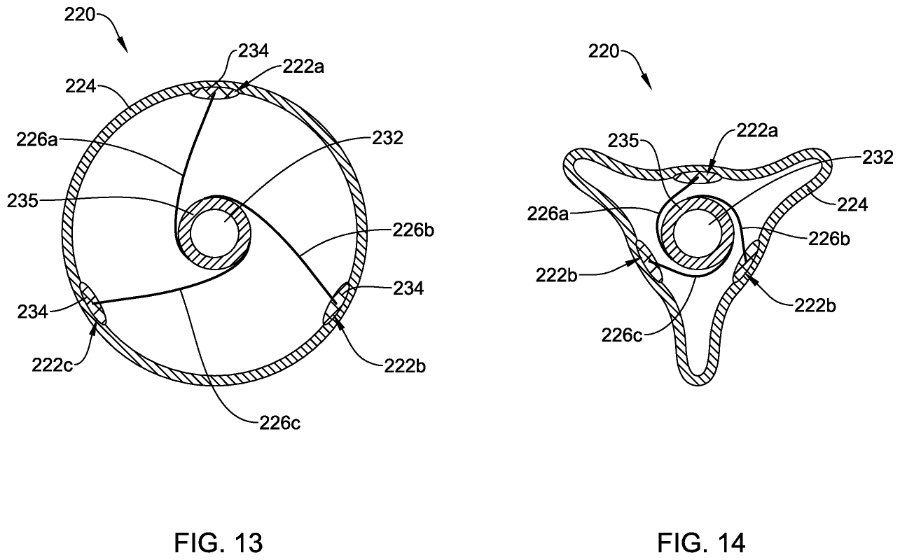

Additionally, as illustrated in FIG. 11, the balloon 220 may include one or more longitudinally extending reinforced portions arranged around the circumference of the balloon 220. For example, the balloon 220 may include one, two, three or more reinforced portions circumferentially arranged around the longitudinal axis of the balloon 220. The balloon 220 illustrated in FIG. 11 includes three reinforced portions 222a, 222b, 222c. The reinforced portion 222c is hidden from view in FIG. 11, but shown in FIGS. 13-14.

In some examples, the reinforced portions 222a, 222b, 222c may include one or more interwoven (e.g., braided, woven, or knitted) filaments 234 attached to and/or embedded in the inner surface of the balloon 220. The filaments 234 may be constructed from a variety of materials. For example, the filaments 234 may be constructed from wire, fiber (e.g., Kevlar® fiber), silk, or other suitable material. In other examples, the reinforced portions 222a, 222b, 222c may include a continuous, flexible strip of material attached to and/or embedded in the inner surface of the balloon 220. Yet in other examples, the reinforced portions 222a, 222b, 222c may include polyimide strips, liquid crystal polymers or other similar materials.

In some examples, each of the reinforced portions 222a, 222b, 222c of FIG. 11 may each include one or more braided or interwoven filaments 234 disposed along an inner surface of the balloon 220. In some examples, the filaments 234 may be braided, wound, wrapped, woven, etc. in a variety of configurations along each of the reinforced to portions 222a, 222b, 222c of the balloon 220. For example, the filaments 234 may be attached directly on to the inner surface of the balloon 220. However, in other examples, the filaments 234 may be extruded with the balloon material such that the filaments 234 are partially embedded into the wall 224 of the balloon 220. In yet other examples, the filaments 234 may be extruded with the balloon material such that the filaments 234 are fully embedded into the wall 224 of the balloon 220. Alternatively, it can be appreciated that the filaments 234 may be adhesively bonded to an inner surface of the balloon 220. The adhesive may include thermoset adhesives that cure either via a chemical reaction or irradiation. In some examples, a polymer coating may be applied over the balloon 220.

FIG. 11 further illustrates that the catheter 200 may further include one, two, three or more tension members 226a, 226b, 226c (e.g., tethers) attached to the reinforced portions 222a, 222b, 222c of the balloon 220 and located within the interior of the balloon 220. For example, FIG. 11 illustrates that the catheter 200 may include a first tension member 226a within the interior of the balloon 220 having a first end attached to a first end of reinforced portion 222a and a second end attached to a second end of reinforced portion 222a. As illustrated in FIG. 11, the portion of the tension member 226a extending between the first end and the second of the tension member 226a may wrap around (e.g., wrap helically around) the inner shaft 232. Similarly, FIG. 11 illustrates that the catheter 200 may include a second tension member 226b within the interior of the balloon 220 having a first end attached to a first end of reinforced portion 222b and a second end attached to a second end of reinforced portion 222b. As illustrated in FIG. 11, the portion of the tension member 226b extending between the first end and the second of the tension member 226b may wrap around (e.g., wrap helically around) the inner shaft 232. As discussed herein, the reinforced portion 222c is hidden from view in FIG. 11, however, FIG. 11 does illustrate the tension member 226c wrapped around (wrapped helically around) the inner shaft 232. In some examples, the inner member may include a reinforcing wire (e.g., braid, mesh, etc.) The reinforcing wire may define a layer of a multi-layer inner shaft 232. Further, in some examples, one or more of the tension members 226a, 226b, 226c, may be integrated with a reinforcing wire of the inner shaft 232. In other words, in some examples the tension members 226a, 226b, 226c may be combined with one or more additional wires to form a reinforced, wire braid in the wall of the inner shaft 232.

Further, it can be appreciated that the tension member 226c may be located within the interior of the balloon 220 and include a first end attached to a first end of the reinforced portion 222c and a second end attached to a second end of the reinforced portion 222c. The reinforced portion 222c and tension member 226c are shown in FIGS. 13-14.

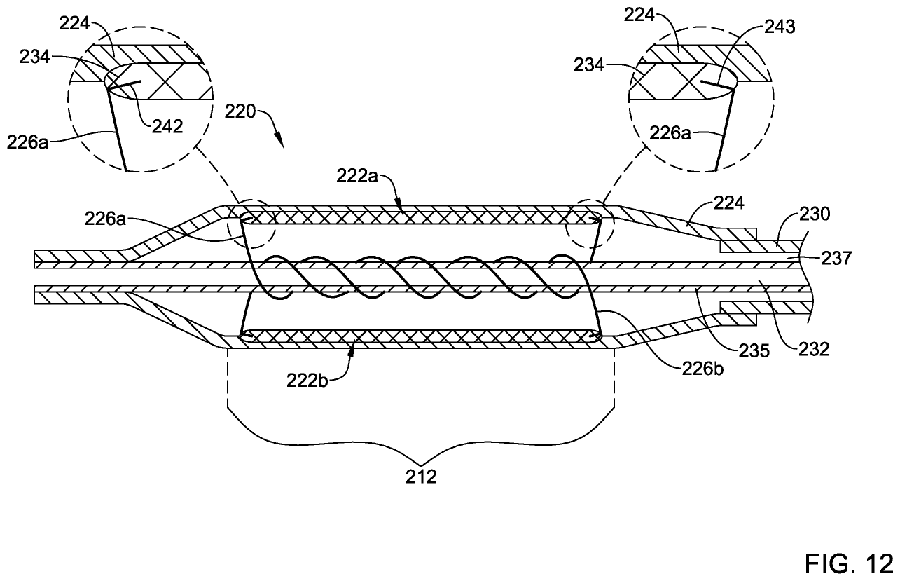

FIG. 12 is a cross-sectional view of the balloon 220 taken along line 12-12 of FIG. 11. As described herein, FIG. 12 illustrates the reinforced portions 222a, 222b (reinforced portion 222c is hidden from view) extending longitudinally along the body portion 212 of the balloon 220. Further, the detailed view of FIG. 12 shows the filaments 234 disposed along the inner surface of the balloon wall 224. As discussed herein, the braided or interwoven filaments 234 may be disposed directly on the inner surface of the balloon wall 224 (e.g., the braided or interwoven filaments 234 may be attached directly onto the inner surface of the balloon wall 224). However, in other examples, the filaments 234 may be partially or fully embedded into the balloon wall 224 (via an extrusion process or other suitable manufacturing method).

As discussed herein, FIG. 12 illustrates the first tension member 226a having a first end attached to a first end of reinforced portion 222a and a second end attached to a second end of reinforced portion 222a. The portion of the tension member 226b extending between the first end and the second of the tension member 226b within the interior of the balloon 220 may wrap around the inner shaft 232. FIG. 12 further illustrates the second tension member 226b having a first end attached to a first end of reinforced portion 222b and a second end attached to a second end of reinforced portion 222b. As illustrated in FIG. 12, the portion of the tension member 226b extending between the first end and the second of the tension member 226b within the interior of the balloon 220 may wrap around the inner shaft 232.

Additionally, the detailed view of FIG. 12 illustrates that a first end 242 of the first tension member 226a may be attached or secured to the braided or interwoven filaments 234 at a first end region of the balloon body 212 and the second end portion 243 of the first tension member 226a may be attached or secured to the braided or interwoven filaments 234 at a second end region of the balloon body 212. In some instances, the first end portion 242 of the first tension member 226a may be embedded in the reinforced portion 222a and the second end portion 243 of the first tension member 226a may be embedded in the reinforced portion 222a. In other instances, the first end portion 242 of the first tension member 226a may be adhesively bonded to the reinforced portion 222a and the second end portion 243 of the first tension member 226a may be adhesively bonded to the reinforced portion 222a, for example. In other examples, the first tension member 226a may extend along the entire length of the reinforced portion 222a. In other words, in some examples, the first tension member 226a may form a loop whereby a portion of the first tension member 226a wraps around the inner shaft 232 and a portion of the first tension member 226a extends along the entire length of the reinforced portion 222a. Similarly, the second tension member 226b may extend along the entire length of the reinforced portion 222b. In other words, in some examples, the second tension member 226b may form a loop whereby a portion of the second tension member 226b wraps around the inner shaft 232 and a portion of the second tension member 226b extends along the entire length of the reinforced portion 222b.

It can be appreciated that attaching the first end portion 242 and the second end portion 243 of the first tension member 226a to the reinforced portions 222a may include passing the braided or interwoven filaments 234 over and/or under both the first end portion 242 and the second end portion 243 of each of the first tension member 226a, thereby securing the first tension member 226a to each of the reinforced portions 222a. It can be further appreciated that the second tension member 226b and the third tension member 226c may be attached to the second reinforced portion 222b and the third reinforced portion 226c, respectively, using the same method of attachment as described to attach the first tension member 226a to the first reinforced portion 222a.

In some examples, the tension members 226a, 226b, 226c may be formed from a high strength fiber (e.g., Kevlar® fiber, Aramid® fiber, Pebax® fiber), wire, silk, nylon, or other suitable material. In yet other examples, the tension members 226a, 226b, 226c may include an elastic member. Further, each of the tension members 226a, 226b, 226c may be designed to impart a radially inward force on each of the reinforced portions 222a, 222b, 222c when the balloon is deflated. Imparting a radially inward force on each of the reinforced portions 222a, 222b, 222c as the balloon is deflated may cause the balloon to refold (e.g., rewrap) in a reduced outer profile configuration prior to the balloon 220 being withdrawn back into an introducer sheath. In other words, imparting a radially inward force on each of the reinforced portions 222a, 222b, 222c as the balloon is deflated may facilitate a refolded balloon 220 which includes a reduced outer profile in a deflated configuration. It can be appreciated a reduced outer profile may be beneficial during withdrawal of the deflated balloon 220 into an introducer sheath. FIGS. 13-14 illustrate the mechanism of imparting an inward radial force on the balloon 220 to refold the balloon 220 as discussed above.

FIG. 13 illustrates a cross-sectional view of the balloon 220 taken along line 13-13 of FIG. 12 with the balloon 220 inflated. FIG. 13 illustrates three longitudinally extending reinforced portions 222a, 222b, 222c circumferentially arranged around the inner shaft 232. FIG. 13 illustrates that the three reinforced portions 222a, 222b, 222c may be equally spaced around the inner shaft 232. However, in other examples, the three reinforced portions 222a, 222b, 222c may be unequally spaced around the inner shaft 232. As discussed herein, the balloon 220 may include one, two, three, four, five, six or more longitudinally extending reinforced portions (e.g., 222a, 222b, 222c) arranged around the inner shaft 232.

FIG. 13 further illustrates the braided or interwoven filaments 234 of each of the reinforced portions 222a, 222b, 222c disposed along the inner surface of the balloon 220. As discussed herein, it can be appreciated from FIG. 13 that a circumferential portion of the balloon wall 224 extends between each of the reinforced portion 222a, 222b, 222c, thereby establishing discrete regions of continuous balloon wall 224 which is devoid of the filaments 234 and/or reinforced portions. In some instances, when fully inflated, the circumferential regions of the balloon wall 224 devoid of the filaments 234 and/or reinforced portions 222a, 222b, 222c may extend radially outward to a greater extent that the portions of the balloon wall 224 directly outward of and secured to the reinforced portions 222a, 222b, 222c. While FIG. 13 illustrates the three reinforced portions 222a, 222b, 222c (each including the filaments 234) disposed along the inner surface of the balloon 220, it is contemplated that, in some examples, the braided or interwoven filaments 234 of each reinforced portion 222a, 222b, 222c may be either partially or fully embedded within the balloon wall 224. In the inflated configurations, the tension members 226a, 226b, 226c may be placed in tension, and thus elastically stretched between the inner shaft 232 and the reinforced portions 222a, 22b, 222c, respectively.

FIG. 14 illustrates the illustrates a cross-sectional view of the balloon 220 in a deflated configuration. Following inflation of the balloon 220, the inflation media may be withdrawn through the outer shaft 230 which may generate a negative pressure (i.e., vacuum) that pulls the inner surface of balloon 220 radially inward (toward the inner shaft 232). However, as discussed herein, each of the tension members 226a, 226b, 226c may be designed to impart a radially inward force on each of the reinforced portions 222a, 222b, 222c. In other words, each of the tension members 226a, 226b, 226c may be designed to pull on the reinforced portion 222a, 222b, 222c to which they are attached. Accordingly, it can be appreciated that the tension members 226a, 226b, 226c may direct the balloon to refold in the configuration shown in FIG. 14, whereby as the tension members 226a, 226b, 226c pull the reinforced portions 222a, 222b, 222c toward the inner shaft 232 in a substantially uniform configuration (e.g., a configuration in which the reinforced portions 222a, 222b, 222c are substantially equally spaced around the circumference of the inner shaft 232). It can be appreciated that imparting a radially inward force on each of the reinforced portions 222a, 222b, 222c encourages a substantially uniform, symmetrical refolding of the balloon 220. Further, the uniform, symmetrical refolding of the balloon 220 may result in a reduced folded profile of the deflated balloon 220, which may enable the balloon to be more easily withdrawn into the introducer sheath that was used for insertion of the balloon 220 into the body.

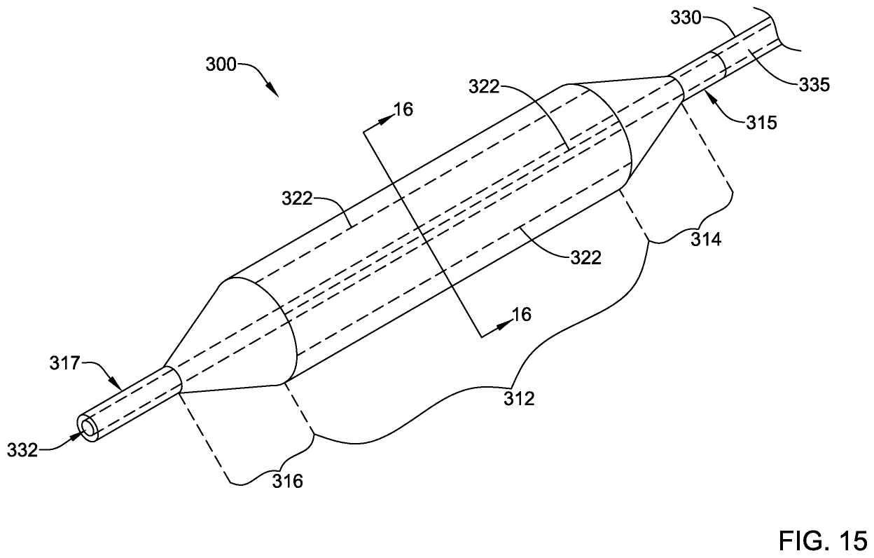

FIG. 15 illustrates another example balloon catheter 300. The balloon catheter 300 may include an expandable medical balloon 320 mounted on or affixed to a catheter shaft, such as a distal end of an outer catheter shaft 330. The medical balloon 320 may be designed to be utilized in a variety of medical procedures, including a valvuloplasty procedure. The catheter shaft 320 may extend from a manifold assembly (not shown) positioned at a proximal end of the catheter shaft 330 and affixed thereto. The balloon 320 may further include a body portion 312, a proximal cone portion 314, a distal cone portion 316, a proximal waist portion 315, and a distal waist portion 317. The body portion 312 may be positioned between the proximal cone portion 314 and the distal cone portion 316, with the proximal waist portion 315 extending proximal of the proximal cone portion 314 and the distal waist portion 317 extending distal of the distal cone portion 316. The balloon 320 may be secured to the outer catheter shaft 330 at the proximal waist 315. It can be appreciated that the catheter shaft of the balloon catheter 300 may also include an inner shaft 332 that may extend within the outer catheter shaft 330, through an inner cavity of the balloon 320 and be secured to the distal waist 317 of the balloon 320.

The inner shaft 332 may include an inner lumen. In at least some embodiments, the inner lumen of the inner shaft 332 may be a guidewire lumen. Accordingly, the catheter 300 may be advanced over a guidewire to the desired location. The guidewire lumen may extend along essentially the entire length of the catheter shaft 330 such that the catheter 300 resembles a traditional “over-the-wire” catheter. Alternatively, the guidewire lumen may extend along only a portion of the shaft 330 so that the catheter 300 resembles a “single-operator-exchange” catheter.

Further, the outer shaft 330 may also include an inflation lumen that may be used, for example, to transport inflation media to and from the balloon 320. When the outer shaft 330 is disposed over the inner shaft 332, the inflation lumen may be defined within the space between the outer surface of the inner shaft 332 and the inner surface of the outer shaft 330.

The balloon 320 may include a balloon wall constructed of one or more layers, wherein each of the layers may be constructed from different balloon materials. For example, the balloon wall 324 of the balloon 320 may include an inner layer and an outer layer, whereby the inner layer is constructed of a lower durometer (e.g., softer) material as compared to the outer layer. This two-layer construction may be referred to as a bi-layer balloon base layer. Further, the inner and outer layer of the bi-layer balloon wall 324 may co-extruded during the manufacturing process of the balloon. Typical balloon materials may include polymer materials, some examples of which are listed herein.

Additionally, as illustrated in FIG. 15, the balloon 320 may include one or more longitudinally extending reinforced portions arranged around the circumference of the balloon 320. For example, the balloon 220 may include one, two, three, four, five, six, seven, eight or more fibers 322 embedded in the wall 324 of the balloon 320. In some examples, the fibers 322 may be formed from a high strength fiber (e.g., Kevlar® fiber, Aramid® fiber, Pebax® fiber), wire, silk, nylon, or other suitable material. The fibers 322 may extend longitudinally along the body 312 of the balloon 320. While FIG. 15 illustrates the fibers 322 extending along the body portion 312 of the balloon, it is contemplated that the fibers 322 may also be embedded within the proximal cone portion 314, the distal cone portion 316 or both the proximal cone portion 314 and the distal cone portion 316.

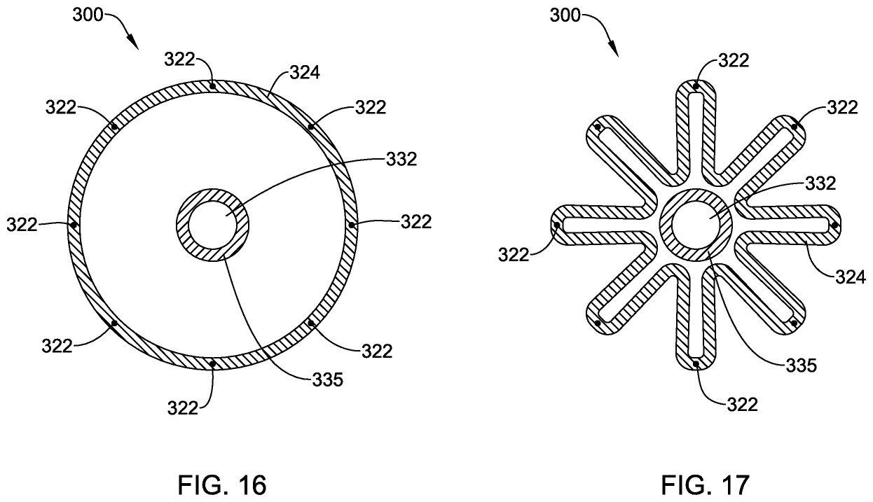

FIG. 16 illustrates a cross-sectional view of the balloon 320 taken along line 16-16 of FIG. 15 with the balloon 320 inflated. FIG. 16 illustrates eight longitudinally extending fibers 322 circumferentially arranged around the inner shaft 332. FIG. 16 illustrates that the eight fibers 322 may be equally spaced around the inner shaft 332. However, in other examples, the fibers 322 may be unequally spaced around the inner shaft 332. As discussed herein, the balloon 320 may include one, two, three, four, five, six, seven, eight, nine, ten or more fibers 322 arranged around the inner shaft 332 and extending longitudinally through the body portion 312, and optionally through or into the proximal cone portion 314 and/or the distal cone portion 316\.

FIG. 16 further illustrates the fibers 322 embedded within the balloon wall 324 of the balloon 320. It can be appreciated from FIG. 16 that a circumferential portion of the balloon wall 324 extends between each of the fibers 322 thereby establishing discrete regions of continuous balloon wall 324 which is devoid of the fibers 322. In some instances, when fully inflated, the circumferential regions of the balloon wall 324 devoid of the fibers 322 may extend radially outward to a greater extent that the portions of the balloon wall 324 having the fibers 322 embedded therein. While FIG. 16 illustrates the fibers 322 fully embedded in the wall 324 of the balloon 320, it is contemplated that, in some examples, the fibers 322 may be partially embedded in the balloon wall 324. It is further contemplated that, in some examples, the fibers 322 may be directly attached to the inner surface of the balloon 320. For example, it is contemplated that the fibers 322 may be adhesively attached, laminated or otherwise attached to the inner surface of the balloon 320.

FIG. 17 illustrates the balloon 320 described in FIG. 16 after vacuum has been pulled to deflate the balloon 320. Further, FIG. 17 illustrates that each of the fibers 322 may provide a reinforced region which is relatively stiff compared to the portions of the balloon wall 324 extending between the fibers 322 (e.g., the portions of the balloon wall 324 which are devoid of the fibers 322). Accordingly, when vacuum is pulled on the balloon 320, the portions of the balloon wall 324 extending between the fibers 322, and thus devoid of the fibers 322, will more readily collapse inwardly towards the inner shaft 332 of the balloon 320, thereby initiating refolding of the balloon 320 into the configuration illustrated in FIG. 17. It can be further appreciated that the resistance to collapse of the fibers 322 of the balloon 320 compared to other portions of the balloon 320 may result in a symmetrical refolding of the balloon 320 during deflation. This symmetrical refolding of the balloon 320 may result in a reduced folded profile of the deflated balloon 320, which may enable the balloon 320 to be more easily withdrawn into the introducer sheath that was used for insertion of the balloon 320 into the body.

Example polymers utilized to manufacture the medical balloons 20, 120, 220, 320 and the various components of the balloons 20, 120, 220, 320 disclosed herein include polymers such as polyethylene terephthalate (PET), polyetherimide (PEI), polyethylene (PE), etc. Some other examples of suitable polymers, including lubricious polymers, may include polytetrafluoroethylene (PTFE), ethylene tetrafluoroethylene (ETFE), fluorinated ethylene propylene (FEP), polyoxymethylene (POM), polybutylene terephthalate (PBT), polyether block ester, polyurethane, polypropylene (PP), polyvinylchloride (PVC), polyether-ester (for example, a polyether-ester elastomer such as ARNITEL® available from DSM Engineering Plastics), polyester (for example, a polyester elastomer such as HYTREL® available from DuPont), polyamide (for example, DURETHAN® available from Bayer or CRISTAMID® available from Elf Atochem), elastomeric polyamides, block polyamide/ethers, polyether block amide (PEBA, for example, available under the trade name PEBAX®), silicones, Marlex® high-density polyethylene, Marlex® low-density polyethylene, linear low density polyethylene (for example, REXELL®), polyetheretherketone (PEEK), polyimide (PI), polyphenylene sulfide (PPS), polyphenylene oxide (PPO), polysulfone, nylon, perfluoro(propyl vinyl ether) (PFA), other suitable materials, or mixtures, combinations, copolymers thereof, polymer/metal composites, and the like. In some embodiments, it may be desirable to use high modulus or generally stiffer materials so as to reduce balloon elongation. The above list of materials includes some examples of higher modulus materials. Some other examples of stiffer materials include polymers blended with liquid crystal polymer (LCP) as well as the materials listed above. For example, the mixture can contain up to about 5% LCP.

It should be understood that this disclosure is, in many respects, only illustrative. Changes may be made in details, particularly in matters of shape, size, and arrangement of steps without exceeding the scope of the disclosure. The disclosure's scope is, of course, defined in the language in which the appended claims are expressed.

What is claimed is:

1. A medical device, comprising:
an outer shaft having a distal end region;
an inner shaft extending within the outer shaft, the inner shaft having an outer surface, a proximal end and a distal end;
a balloon including a distal waist coupled to the distal end of the inner shaft, a proximal waist coupled to the distal end of the outer shaft, an inner surface and a wall;
a first interior partition panel disposed within the balloon, wherein the first interior partition panel is coupled to both the outer surface of the inner shaft and an inner surface of the balloon; and
a second interior partition panel disposed within the balloon, wherein the second interior partition panel is coupled to both the outer surface of the inner shaft and an inner surface of the balloon;
wherein the first interior partition panel is circumferentially spaced away from the second interior partition panel.

2. The medical device of claim 1, wherein the balloon is configured to shift between an inflated configuration and a deflated configuration, and wherein the balloon includes a first inflation chamber positioned between the first interior panel and the second interior panel.

3. The medical device of claim 1, wherein the balloon is configured to be inflated to a first pressure, and wherein the balloon wall between the first panel and the second panel has a diameter at the first pressure and wherein a portion of the balloon wall attached to the first panel has a diameter at the first pressure, and wherein the diameter of the balloon wall between the first panel and the second panel is greater than the diameter of the portion of the balloon wall attached to the first panel at the first pressure.

4. The medical device of claim 1, wherein both of the first panel and the second panel extend along a longitudinal axis of the inner shaft.

5. The medical device of claim 1, wherein the first panel is attached to the inner shaft along a first longitudinal attachment region, and wherein the first panel is substantially perpendicular to a tangent line passing through a point on the first attachment region along the inner shaft.

6. The medical device of claim 1, wherein the first panel is attached to the inner shaft along a first longitudinal attachment region, and wherein the first panel forms an acute angle relative to a tangent line passing through a point on the first attachment region along the inner shaft.

7. The medical device of claim 6, wherein the second panel is attached to the inner shaft along a second longitudinal attachment region, and wherein the second panel forms an acute angle relative to a tangent line passing through a point on the second attachment region along the inner shaft.

8. The medical device of claim 1, wherein the balloon is configured to shift between an inflated configuration and a deflated configuration, and wherein the portions of the balloon wall adjacent a portion of the balloon wall attached to the first panel and the second panel are configured to collapse onto the first panel and the second panel in the deflated configuration.

9. The medical device of claim 8, wherein the second panel is configured to fold onto the first panel in the deflated configuration.

10. The medical device of claim 2, wherein the balloon further comprises a third interior partition panel, wherein the third interior partition panel is attached to the outer surface of the inner shaft and an inner surface of the balloon, wherein the third interior partition panel is circumferentially spaced from the second interior partition panel, and wherein the balloon includes a second inflation chamber positioned between the third interior panel and the second interior panel, and wherein the first inflation chamber is configured to be inflated independent of the second inflation chamber.

11. A medical device, comprising:
an outer shaft having a distal end region;
an inner shaft extending within the outer shaft, the inner shaft having an outer surface, a proximal end and a distal end;
a balloon including a distal waist secured to the distal end of the inner shaft, a proximal waist secured to the distal end of the outer shaft, a wall, and a body portion positioned between the distal waist and the proximal waist;
a first reinforced region positioned along the body portion; and
a first tension member coupled to the first reinforced region and the inner shaft.

12. The medical device of claim 11, wherein the first reinforced region includes a first plurality of filaments.

13. The medical device of claim 12, wherein the first tension member includes a first end region, a second end region and a medial region, and wherein the medial region is wound around the outer surface of the inner shaft.

14. The medical device of claim 13, wherein the first reinforced region includes a distal end and a proximal end, and wherein the first end region of the first tension member is coupled to the distal end of the reinforced region and wherein the second end region of the first tension member is coupled to the proximal end of the reinforced region.

15. The medical device of claim 14, wherein the first tension member includes a fiber.

16. The medical device of claim 15, wherein the balloon is configured to shift between an inflated configuration and a deflated configuration, and wherein the first tension member imparts a radially inward force on the balloon as the balloon shifts between the inflated configuration and the deflated configuration.

17. The medical device of claim 15, wherein the balloon is configured to shift between an inflated configuration and a deflated configuration, and wherein inflation of the balloon imparts a tensile force on the first tension member.

18. The medical device of claim 11, further comprising a second reinforced region positioned along the body portion and a second tension member, wherein the second tension member is coupled to the second reinforced region and the inner shaft.

19. The medical device of claim 18, wherein the second tension member is wound around the inner shaft.

20. A method of using a balloon catheter, comprising the steps of:
advancing a balloon catheter through a body vessel to a target site, the balloon catheter comprising:
an outer shaft having a distal end region;
an inner shaft extending within the outer shaft, the inner shaft having an outer surface, a proximal end and a distal end;
a balloon including a distal waist coupled to the distal end of the inner shaft, a proximal waist coupled to the distal end of the outer shaft, an inner surface and a wall;
a first interior partition panel disposed within the balloon, wherein the first interior partition panel is coupled to both the outer surface of the inner shaft and an inner surface of the balloon; and
a second interior partition panel disposed within the balloon, wherein the second interior partition panel is coupled to both the outer surface of the inner shaft and an inner surface of the balloon;
wherein the first interior partition panel is circumferentially spaced away from the second interior partition panel; and
inflating the balloon, whereby the balloon engages the target site;
deflating the balloon; and
withdrawing the balloon catheter from the body vessel.
